(19) United States
(12) Patent Application Publication
(10) Pub. No.: US 2024/0093187 A1
(43) Pub. Date: Mar. 21, 2024

(54) SYSTEM AND METHOD FOR TISSUE REJUVENATION AND PROTEIN SYNTHESIS BY PROGRAMMED RIBOSOMES

(71) Applicant: Arthur Mikaelian, Beverly Hills, CA (US)

(72) Inventor: Arthur Mikaelian, Beverly Hills, CA (US)

(21) Appl. No.: 18/467,595

(22) Filed: Sep. 14, 2023

Related U.S. Application Data
(60) Provisional application No. 63/375,742, filed on Sep. 15, 2022.

(51) Int. Cl. C12N 15/11; C12N 13/00

(52) U.S. Cl. CPC C12N 15/111; C12N 13/00; C12N 2320/53

(57) ABSTRACT

A technology that may be used to program ribosomes to synthesize specific proteins and rejuvenate tissues and include the ability to modulate or program the function of ribosomes towards a specific desired function, such as for use with tissue rejuvenation and the production of missing or mutated proteins.

### CROSS-REFERENCE TO RELATED APPLICATIONS

This U.S. Non-Provisional patent application claims priority from and the benefit of U.S. Provisional Patent Application No. 63/375,742, filed on Sep. 15, 2022, titled “TISSUE REJUVENATION BY PROGRAMING RIBOSOMES,” by sole inventor Arthur Mikaelian, the contents of which are expressly incorporated herein by this reference as though set forth in their entirety.

### FIELD

The present disclosure is directed to a system and method to modulate and program ribosomes to synthesize specific proteins and encourage tissue rejuvenation.

### BACKGROUND

Genetic information is an intricate archiving system encoded within DNA, serving as the fundamental blueprint that cells harness to execute the myriad functions essential to life.

Within the intricate confines of each cell, specialized catalysts diligently seek out the pertinent information from this genomic archive, processing it to construct novel proteins. These proteins not only form the foundational structures of the cell but also orchestrate the numerous biochemical reactions therein. Some are even destined for export, playing pivotal roles in neighboring or distant cells.

Intriguingly, while every cell of a multicellular organism shares an identical genetic reservoir, functionally distinct cells exhibit remarkable selectivity. They employ unique sets of catalysts to decipher only the relevant portions of these genomic instructions, thus ensuring precise protein synthesis tailored to their role and needs.

The pivotal journey from DNA to protein commences with transcription. The cell's genetic script is transcribed into a transient format: the messenger RNA (mRNA). Assisted by a suite of enzymes known as RNA polymerases, these mRNA molecules then embark on their mission, bearing the encoded instructions to cellular factories called ribosomes.

Ribosomes stand as the cellular nexus where protein synthesis unfolds. The abundance of ribosomes in a cell offers a direct glimpse into its protein-synthesizing fervor. For instance, cells in the throes of rapid proliferation often teem with ribosomes.

Structurally, ribosomes are intricate assemblies of ribosomal RNA (rRNA) molecules and a medley of proteins, partitioned into two distinct subunits. Within this molecular marvel, rRNA choreographs the intricate dance of protein synthesis.

The advent of ribosome-centric therapeutic strategies may herald a new era in medicine. Contrasted with vector-induced gene therapies, ribosomal approaches promise diminished immunogenicity. Moreover, they offer a potential longevity advantage over mRNA therapies.

A gamut of diseases can trace their origins to anomalies in ribosome biogenesis or function. An emergent perspective, however, proposes harnessing ribosomes in novel therapeutic modalities targeting DNA-related diseases.

Traditional gene therapy paradigms primarily focus on the introduction, modulation, or silencing of genetic material. However, ribosome-targeted therapies have emerged as a novel approach. Their potential spans from treating specific diseases to innovative anti-aging interventions.

Beyond disease rectification, the realm of anti-aging research has been abuzz with the tantalizing prospect of telomere extension. By programming ribosomes to synthesize telomerase, it's conceivable to decelerate or even reverse certain cellular aging processes.

Furthermore, the strategic programming of ribosomes also paves the way for innovative vaccine designs. By enabling the synthesis of specific antigenic proteins without the accompanying undesired elements from pathogens, it offers the potential to develop vaccines with minimal to no side effects. Such an approach could revolutionize the immunization landscape, offering safer alternatives to current methodologies.

Alongside vaccines, the realm of antibiotics also stands to benefit. Programmed ribosomes could be harnessed to synthesize next-generation antibiotics that specifically target pathogenic bacteria without affecting the beneficial microflora or eliciting common antibiotic side effects. This could pave the way for more effective and safer therapeutic agents, especially in an era where antibiotic resistance is a mounting concern.

Ribosome-targeted therapies thus emerge as a noble approach for a new generation of treatments. Their potential spans from treating specific diseases with defects in ribosome biogenesis and function to innovative anti-aging interventions, and even to the realms of vaccine and antibiotic development.

Thus, there exists the need for a way to modulate and program ribosomes to synthesize specific proteins in human cells for tissue regeneration.

### SUMMARY

To minimize the limitations in the cited references, and to minimize other limitations that will become apparent upon reading and understanding the present specification, the present specification discloses a polarized scorpion venom solution useful for the treatment of Vitiligo and other diseases associated with pigment loss.

One embodiment may be a method of generating a polypeptide may comprise the steps: providing a sequence of polypeptide; converting the sequence of polypeptide into a series of a plurality of mRNA codon sequences; labeling each of the mRNA codon sequences and identifying the mRNA codon sequence based on an order in which the mRNA codon sequences are structured, such that each of the mRNA codon sequences are identified as an nth mRNA codon sequence, wherein n refers to the order in which the mRNA codon sequences are situated pursuant to the series; identifying a frequency corresponding to each of the mRNA. codon sequences; emitting a series of electromagnetic signals based on the frequency, such that a stimulated ribosome synthesizes the polypeptide as if a physical mRNA having the series of the plurality of mRNA codon sequences; and generating the polypeptide by the stimulated ribosome. The polypeptide may be a therapeutic protein. The sequence may be designed by humans. The method of generating a polypeptide further may comprise the step of tissue regeneration by the polypeptide. The method of generating a polypeptide further may comprise the step of increasing lifespan of certain cells by the polypeptide. The method of generating a polypeptide further may comprise the step of lengthening telomeres by the polypeptide.

Another embodiment may be a device for stimulating ribosomes, comprising: an electromagnetic signal transmitter; wherein the electromagnetic signal transmitter is configured to emit an electromagnetic signal to stimulate a ribosome into synthesizing a polypeptide; wherein the polypeptide is configured to be translated by the ribosome from a series of codons; wherein attributes of the electromagnetic signal are calculated based on an analysis of the series of codons; and wherein the analysis is conducted by converting the molecular properties of individual nucleotides into a frequency, wherein the attributes comprise the frequency.

Another embodiment may be a method of programming ribosomes to synthesize specific proteins by altering their RNA sequence. The programmed ribosomes may be configured to inhibit production of undesirous proteins; wherein the undesirous proteins may promote tumor growth.

In one embodiment, the addition of ribosomes may increase cell production, or prolong cell life.

In one embodiment, the addition of ribosomes may enhance ATP production in certain cells.

In one embodiment, ribosomes may be exposed to external stimulus, such as waves of specific amplitude or frequency, in order to selectively activate certain ribosomes.

The present disclosure may be applied to, but is not limited to, genetic, neurology, endocrinology, hematology, immunology, and dermatology-related diseases.

The present disclosure may also be applied to the anti-aging field of medicine and cosmetology.

In one embodiment, the present disclosure may be used to produce specific proteins within specific cells.

The present disclosure may allow for programming ribosomes to produce specific proteins that may be deficient or mutated in the human body which may offer one possible treatment for disease conditions.

The present disclosure uses ribosomes to produce proteins and/or rejuvenate tissues. In one embodiment, ribosomes isolated from human kidney fibroblasts may be placed in solution to enhance the growth of human kidney fibroblasts and extend the life of the human kidney fibroblasts.

Introducing a solution that includes ribosomes may increase ATP production in human kidney fibroblasts by 95% more than a control culture of human kidney fibroblasts.

In one embodiment, ribosomes from human kidney fibroblasts cultured with human dermal fibroblasts induced the production of erythropoietin.

In one embodiment, when ribosomes isolated from human dermal fibroblasts are then cultured with human dermal fibroblast and erythropoietin mRNA, human dermal fibroblasts may synthesize erythropoietin.

In one embodiment, “METHOD AND TECHNOLOGY OF RESONANT FREQUENCIES FOR ATOMIC AND SUBATOMIC PARTICLE EXCITATION” may be used to simulate the frequency of codons by generating specific electromagnetic frequencies to simulate the codon of mRNA and thus program ribosomes to produce specific proteins and rejuvenate tissues.

The present disclosure pertains to the interdisciplinary domain of molecular biology, biochemistry, and regenerative medicine. The primary focus lies in harnessing the molecular machinery of cells—specifically ribosomes—to achieve targeted protein synthesis and foster tissue rejuvenation and synthesis of desired proteins. This approach utilizes fundamental principles of cellular biology and ribosomal mechanics for therapeutic applications related to tissue degeneration, genetic disorders, and protein deficiencies, which may also play a role in personalized medicine.

In one embodiment, ribosomes may be modulated and programmed in precise ways to enable specific and targeted synthesis of desired proteins. Moreover, this technique capitalizes on the inherent capability of ribosomes, extending it toward potential applications in tissue rejuvenation. By manipulating the ribosomal activities at a molecular level, the invention sets the stage for breakthrough advancements in regenerative medicine, offering prospects for targeted tissue repair and age-related cellular rejuvenation processes.

One embodiment may be a programmed ribosome configured for synthesizing proteins based on therapeutic, regenerative, or biotechnological needs.

Another embodiment may be used in treating, preventing, or managing a range of diseases, disorders, or conditions.

Another embodiment may be a method for controlling protein synthesis: Employing programmed ribosomes to enhance, inhibit, or modify the synthesis of specific proteins within a cell.

Another embodiment may be use of the programmed ribosome for applications related to tissue regeneration, cellular longevity, or anti-aging interventions.

Another embodiment may be a system for producing and delivering programmed ribosomes: Incorporating means for bulk production and targeted delivery to cells or tissues.

Another embodiment may be use of the programmed ribosome in conjunction with other biomedical techniques, including gene editing, to facilitate or ensure desired cellular outcomes.

Another embodiment may be a composition comprising programmed ribosomes: Suitable for various delivery methods and combined with a pharmaceutically acceptable carrier.

Another embodiment may be a method employing programmed ribosomes: For research, including building databases of sequences or developing software algorithms for ribosomal programming.

Another embodiment may be a kit comprising programmed ribosomes: Accompanied with instructions or materials for its use in various therapeutic, research, or biotechnological applications.

It is an object of the present disclosure to overcome the limitations of the prior art.

Additional embodiments of the disclosure will be understood from the detailed description of the disclosure.

### BRIEF DESCRIPTION OF THE DRAWINGS

The drawings show illustrative embodiments, but do not depict all embodiments. Other embodiments may be used in addition to or instead of the illustrative embodiments. Details that may be apparent or unnecessary may be omitted for the purpose of saving space or for more effective illustrations. Some embodiments may be practiced with additional components or steps and/or without some or all components or steps provided in the illustrations. When different drawings contain the same numeral, that numeral refers to the same or similar components or steps.

FIG. 1 shows that human kidney fibroblasts may grow in culture for few days (Day 11), but then die by (Day 14).

FIG. 2 shows that the adding ribosomes-containing solution may allow for growth of human kidney fibroblasts, whereas adding non-ribosome containing solution may allow human kidney fibroblasts to die.

FIG. 3 shows that ribosome-containing solution may enhance ATP production in human kidney fibroblasts.

FIG. 4 shows that ribosomes-containing solution isolated from kidney fibroblasts and cultured with human dermal fibroblasts may enable the latter cells to produce erythropoietin.

FIG. 5 is an illustration of one embodiment of a ribosome synthesizing a polypeptide through translating an mRNA.

FIG. 6 is an illustration of one embodiment of a ribosome synthesizing a polypeptide through receiving an electromagnetic signal.

FIG. 7 is an illustration of one embodiment of a ribosome being activated by an electromagnetic signal generating device.

### DETAILED DESCRIPTION

In the following detailed description of various embodiments of the disclosure, numerous specific details are set forth in order to provide a thorough understanding of various aspects of one or more embodiments of the disclosure. However, one or more embodiments of the disclosure may be practiced without some or all of these specific details. In other instances, well-known methods, procedures, and/or components have not been described in detail so as not to unnecessarily obscure aspects of embodiments of the disclosure.

While multiple embodiments are disclosed, still other embodiments of the present disclosure will become apparent to those skilled in the art from the following detailed description, which shows and describes illustrative embodiments of the disclosure. As will be realized, the disclosure is capable of modifications in various obvious aspects, all without departing from the spirit and scope of the present disclosure. Accordingly, the figures, and the detailed descriptions thereof, are to be regarded as illustrative in nature and not restrictive. Also, the reference or non-reference to a particular embodiment of the disclosure shall not be interpreted to limit the scope of the disclosure.

As used herein, the terms “approximately” and “about” generally refer to a deviance of within 5% of the indicated number or range of numbers. In one embodiment, the term “approximately” and “about”, may refer to a deviance of between 1-10% from the indicated number or range of numbers.

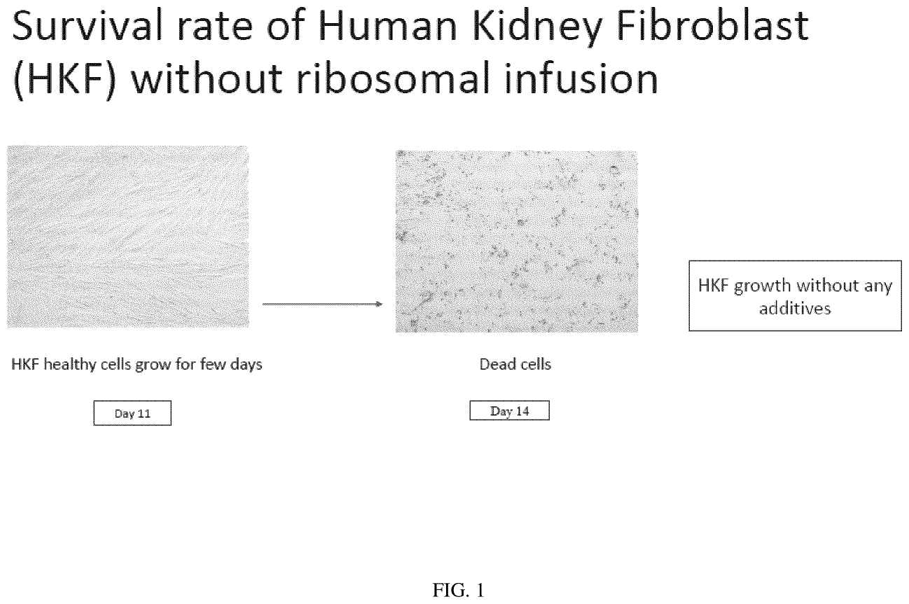

FIG. 1 shows that human kidney fibroblasts may grow in culture for few days (Day 11), but then die by (Day 14).

The present disclosure utilizes ribosomes as dynamic protein factories, earmarked for driving tissue rejuvenation. Through methodological experimentation, in one embodiment, ribosomes were extracted from human kidney fibroblasts and introduced to an optimal solution, were observed to amplify the growth longevity of these fibroblasts substantially. Quantitatively, in one embodiment, this effect led to a marked 95% uptick in ATP production, which may underscore the profound bioenergetic implications of our invention.

In one embodiment, these specialized ribosomes were made to interface with human dermal fibroblasts, the interaction resulted in an unexpected but highly beneficial outcome a surge in erythropoietin production. This may have substantial therapeutic implications, such as in the context of wound healing and tissue repair.

In one embodiment, an electromagnetic signal generator may be used to influence, modulate, or stimulate a ribosome. In one embodiment, precise electromagnetic signals may allow for tailored protein synthesis, by having the electromagnetic signals generated by the electromagnetic signal generator simulate or mimic the presence of a specific mRNA sequence for use with the ribosome.

First, deliberate dissociation of mRNA from ribosomal complexes may be initiated, which may allow for the retention of ribosomal structural and functional integrity, while also preparing ribosomes for the impending programming phase.

Second, an electromagnetic signal generator may be used to emit precisely calibrated electromagnetic frequencies. Through refinement, these frequencies may be designed to emulate the unique signaling attributes of mRNA. In some embodiments, the mRNA emulated may be human.

As these frequencies (or signals) interact with the mRNA-devoid ribosomes, they stimulate a programming sequence, replicating the protein synthesis directive that mRNA typically undertakes. This paradigm shift may not only bypass the need for human mRNA but also significantly reduces any potential immunological backlash. Traditional therapeutic methods, which often rely on exogenous mRNA, are frequently plagued by immunological challenges.

On the one hand, an electromagnetic signal generator may be used to enhance the accuracy and efficiency of ribosomal programming. On the other hand, the present disclosure may considerably elevate the safety profile of these interventions. This dual advantage may be indicative of significant breakthroughs for ribosomal therapeutics with potentially far-reaching clinical applications.

### Protocol One

### Materials and Methods

1. Cell Lines:
Human Dermal Fibroblasts (HDF): Primary Dermal Fibroblast; Normal, Human, Adult (HDFa) (ATCC® PCS-201-012™)
Human Kidney Fibroblasts: Sourced from either Accegen Biotechnology (ABC-TC5519) or Cell Biologics (H-6016).

2. Cell Culture Media:

### Human Derma Fibroblast Maintenance Media:

Fibroblast Basal Medium (ATCC® PCS-201-030TH)
Fibroblast Growth Kit-Low serum (ATCC® PCS-201-041™)

### Human Kidney Fibroblast Maintenance Media:

Provided by Accegen Biotechnology

3. Cell Culture Maintenance
PBS (ThermoFisher Catalog number: 20012043)
Trypsin-EDTA for Primary Cells (ATCC® PCS-999-003™)
Penicillin-Streptomycin-Amphotericin B Solution (ATCC® PCS-999-002™)

### Flask and Plates for Tissue Culture:

T25, T75, 4-well culture plates, and 96-well culture plates.

### Filters:

0.2 μm syringe-mediated filters
Amicon Ultrafilter-15 100 KD (Z740208-8EA Sigma-Millipore)
Amicon Ultrafilter-15 3 KD (Z740199-8EA Sigma-Millipore)

4. Reagents and Buffers:

### Reagents:

Igepal CA-630 (NP-40) (18896-50ML, Sigma-Millipore)
m-β-cyclodextrin (C4555-1G, Sigma-Millipore)

### Buffers:

Buffer A: 15 mM Tris (pH 7.5), 6 mM MgCl2, 300 mM NaCl
Buffer B: 150 KCL, 5 mM Mg(OAC)2, 20 mM HEPES, 1 mM DTT, 10 mM NH4CL (pH 7.4)
Note: Both buffers should be supplemented with RNAsin-Plus RNAase inhibitor (Promega) at a final concentration of 0.2 U/μl.

5. Ribosome Preparation:
A. Reach cell confluency of 85-90%. Trypsinize the cells to prepare a cell pellet of human kidney fibroblasts (≥10×10⁶) and add to it a chilled 2-ml volume of Buffer A+0.5% Igepal, vortex and incubate for 15 minutes on ice. Then centrifuge at 6000×g for 30 min at 4° C. and collect the supernatant.
Using the Amicon Ultrafilter 100 KD, centrifuge the supernatant at 6000×g for 30 min at 4° C. The upper filtrate contains the ribosomes, and the lower filtrate are non-ribosomal proteins. Do a protein measurement for both the upper and the lower filtrate and adjust both to 1 μg/μl. Run each filtrate on Amicon Ultrafilter 3 KD and centrifuge centrifuged at 6000×g for 30 min at 4° C. Then resuspend the upper filtrate with Buffer B (Ribosome-containing solution, RCS), and the lower filtrate is resuspended with Buffer B (non-Ribosome-containing solution) (control solution, NRCS).
RCS and NRCS can be stored at −80 C for future use.

B. mRNA Separation
Dynabeads® mRNA DIRECT™ Kit (Ambion Catalog numbers 61011 and 61012)
Introduction: The mRNA content of cells and tissues varies depending on the source of the material and RNA expression levels at the time of tissue/cell harvest. Dynabeads® mRNA DIRECT™ Kit protocols can be scaled up or down to suit specific sample sources and quantities.
Protocol as directed by the supplier.

6. Detection Systems:
OCT4 (Stem Cell Maintenance Marker): ThermoFisher (MA 104) and secondary Ab (35502).
Product Description
Invitrogen™
PSC Immunocytochemistry Kits contain sets of primary and secondary antibodies along with ready-to-use buffers to enable convenience.
immunocytochemistry characterization of human pluripotent stem cells (hPSC). The primary antibodies included in these kits target well-established hPSC markers (OCT4, SOX2, SSEA4, and/or TRA-1-60) and were carefully selected to help ensure excellent performance in immunocytochemistry applications
Collagen I immunohistochemistry: Collagen I Antibody (PAS-95137) for IF ThermoFisher, supplemented with biotinylated goat anti-rabbit IgG and Strepavidin-Biotin-Complex/DABI.
Human Erythropoietin Quantification Kits: Either Human Erythropoietin Quantikine IVD ELISA Kit from R&D systems or Human Erythropoietin ELISA Kit (EPO) (ab119522).

Abcam's Erythropoietin (EPO) Human in vitro ELISA (Enzyme-Linked Immunosorbent Assay) kit is designed for accurate quantitative measurement of Human Erythropoietin concentrations in cell culture supernatant, serum and plasma (EDTA, citrate, heparin).

For therapeutic applications, the primary step involves a meticulous selection of ribosomal types, ensuring their alignment with the therapeutic objectives. This is predicated on understanding the functional nuances of ribosomal variants and their compatibility with the intended therapeutic regimen.

Once the ribosomal type is determined, the next phase involves the acquisition of specific cells tailored for the targeted therapy. This choice is pivotal, as cellular attributes can significantly influence the efficiency of ribosomal protein synthesis and, consequently, the therapeutic outcomes.

The procured cells are then cultured in specialized growth media optimized for proliferation. Continuous monitoring of the cell culture environment, including factors like pH, nutrient availability, and temperature, ensures an optimal yield. The cells undergo a series of passages, wherein they are transferred to fresh media periodically. This process, if done under stringently controlled conditions, amplifies the cell population without compromising their physiological characteristics.

Upon achieving the desired cell concentration, a series of centrifugation and purification steps are employed. The initial rounds of centrifugation help in the sedimentation of whole cells and larger debris. The subsequent supernatant, enriched in ribosomes, undergoes further refinement processes. Advanced ultra-centrifugation is applied to separate the ribosomes from mRNA and other cellular micro-components. It's imperative that this isolation process is thorough to prevent as much as possible mRNA presence,

By the end of this thorough process, we are left with a pure ribosomal preparation, poised for therapeutic application, ensuring maximal efficacy and minimal side effects.

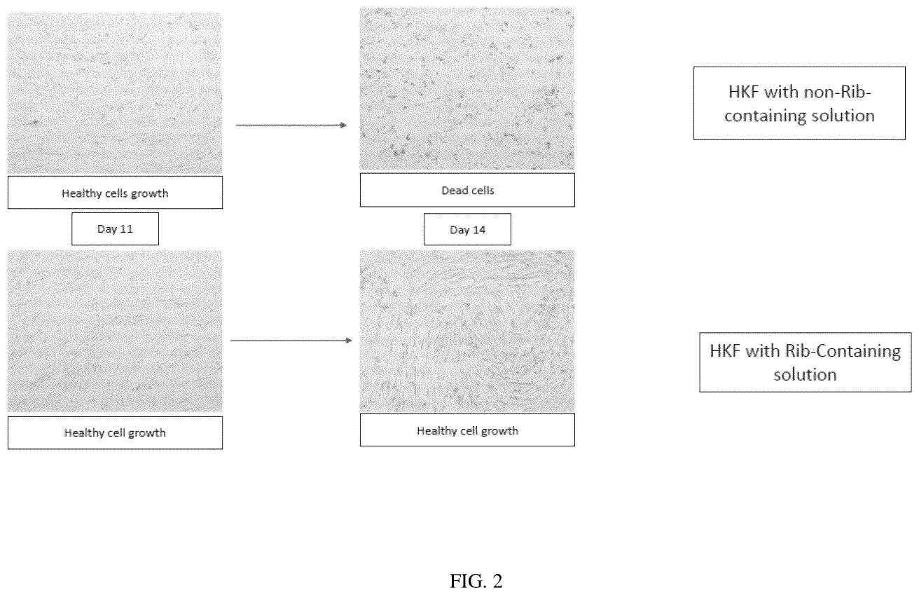

FIG. 2 shows that the adding ribosomes-containing solution may allow for growth of human kidney fibroblasts, whereas adding non-ribosome containing solution may allow human kidney fibroblasts to die.

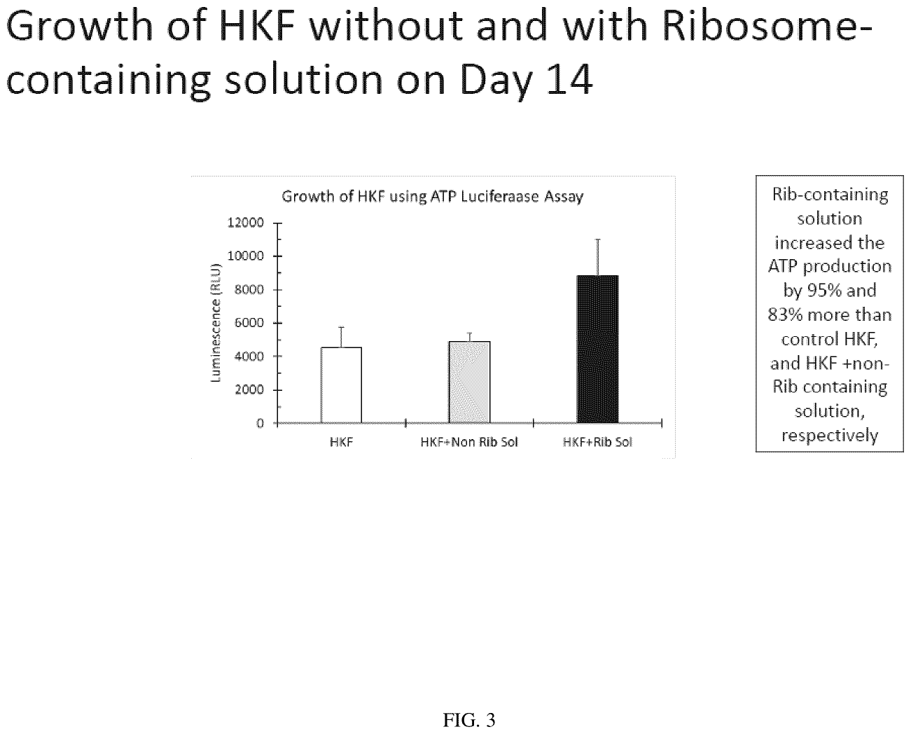

FIG. 3 shows that ribosome-containing solution may enhance ATP production in human kidney fibroblasts.

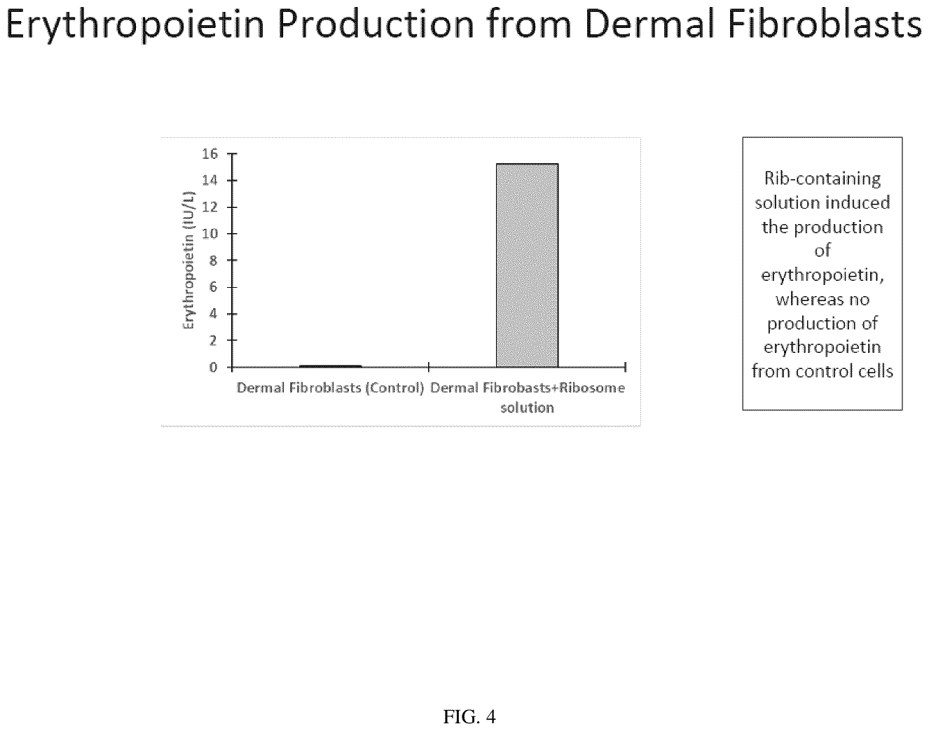

FIG. 4 shows that ribosomes-containing solution isolated from kidney fibroblasts and cultured with human dermal fibroblasts may enable the latter cells to produce erythropoietin.

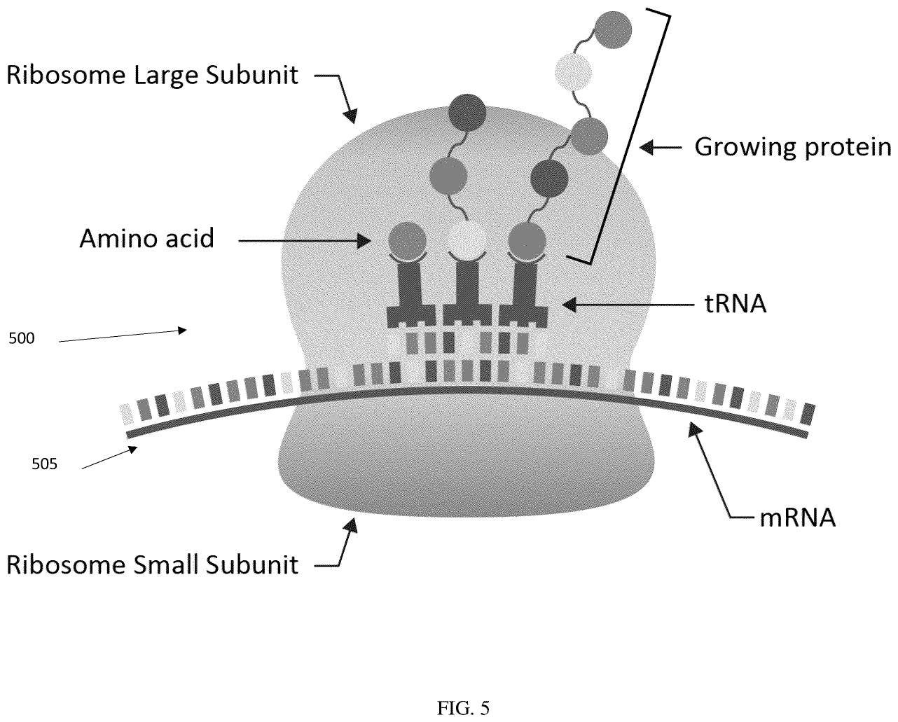

FIG. 5 is an illustration of one embodiment of a ribosome synthesizing a polypeptide through translating an mRNA into a growing protein. The translation method used by the ribosome 500 may be considered a well-known method in the art for translating mRNA 505 to growing protein.

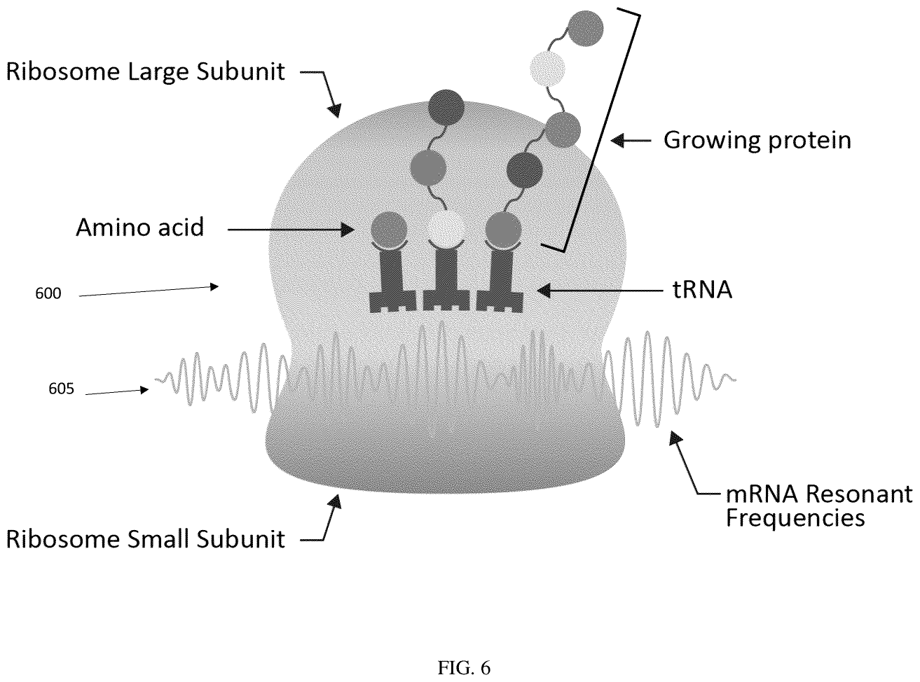

FIG. 6 is an illustration of one embodiment of a ribosome synthesizing a polypeptide through receiving an electromagnetic signal. The translation method used by the ribosome 600 may essentially replace the use of mRNA with the electromagnetic signal 605.

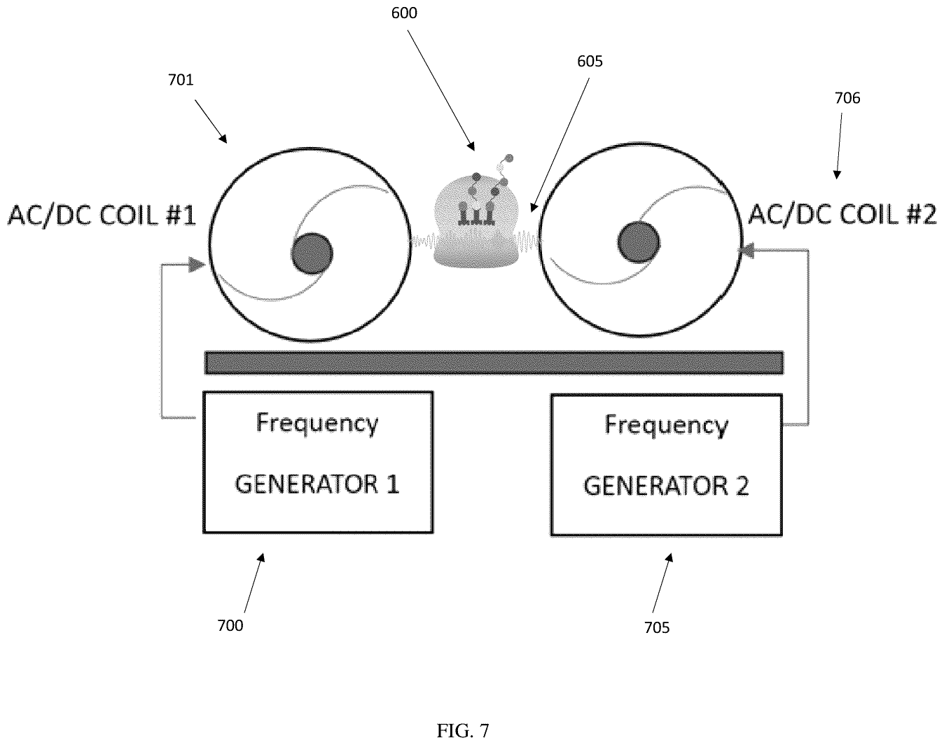

FIG. 7 is an illustration of one embodiment of a ribosome being activated by an electromagnetic signal generating device. As shown in FIG. 7, a first frequency generator 700 and second frequency generator 705 may cause the electromagnetic signal 605 to be generated by a first coil 701 and second coil 706, respectively. The electromagnetic signal 605 may cause the ribosome 600 to produce a polypeptide or protein by simulating the effects of mRNA engaging the ribosome 600.

### Quantum Programming of the Ribosomes

Intrinsic Resonance Characteristics of Particles: The atomic and subatomic realm is governed by the laws of quantum mechanics, presenting a fascinating landscape where particles exhibit wave-like and particle-like behavior simultaneously. Within this context, each atomic and subatomic entity—be it an electron, proton, or even more massive nuclei—exhibits a specific resonance frequency. This frequency is determined by an interplay of the particle's intrinsic quantum mechanical properties, such as its spin, charge, and mass, as well as the spatial configuration and boundary conditions in which it is situated.

In the context of the method disclosed herein, the initiation codon for mRNA, represented as AUG, can be translated into a frequency representation as f(A)(U)(G). Here, “f” denotes the specific resonance frequency associated with each individual codon. This approach allows for the mimicking of the nucleotide sequence using precise frequency patterns.

5′-AUGCGUACGUAGCUACGUACGUACG
UAGCUAGCUAGCUAGC . . . - 3′

### Here:

f1(AUG) represents the frequency pattern for the AUG codon.
f2(CGU) represents the frequency pattern for the CGU codon . . . . and so on. This representation condenses the mRNA sequence into blocks of frequencies that correspond to individual codons, allowing for more efficient interpretation or interaction based on a method disclosed herein.

### Example Sequences:

5′-AUGCGUACGUAGCUACGUACGUACG
UAGCUAGCUAGCUAGCUAA-3′

Using the method disclosed herein representation where each “f” represents a triplet codon:
f1(AUG) f2(CGU) f3(ACG) f4(UAG) f5(CUA) f6(CGU) f7(ACG) f8(UAG) f9(CUA) f 10(GCU) f11(UAA)

### Here:

f1(AUG) is the start codon, signifying the initiation of translation.
f11(UAA) is the stop codon, signifying the termination of translation.
   Note: The given sequence and representation are illustrative. In an actual biological context, the mRNA would have more diverse codons, corresponding to a sequence that encodes a specific protein.

### Method for Stimulating Ribosomal Function Using Resonance Frequencies

### 1. Resonance Frequency Derivation:

After the transcription process produces an mRNA molecule, this sequence is fed into specialized software.
The software calculates the resonance frequency for each nucleotide (or codon) in the mRNA sequence using the Mikaelian method. This involves a theoretical conversion of the molecular or electronic properties of the nucleotide into a specific frequency, based on the nucleotide's mass, charges, protein, neutron, electron quantity, and some additional properties.

### 2. Frequency Generation:

The derived frequencies are input into the Mikaelian Resonance Generator, a device capable of producing precise electromagnetic waves corresponding to the resonance frequencies of the individual nucleotides or codons.
These frequencies are modulated in sequence, corresponding to the order of nucleotides in the mRNA molecule.

### 3. Targeting the Ribosome:

The ribosomes to be stimulated are exposed to the output of the Mikaelian Resonance Generator.
The generated resonance frequencies penetrate the ribosomes. These frequencies mimic the interaction of the tRNA or other initiation factors with the ribosome, prompting it to begin translation.

### 4. Stimulation of Translation:

Upon receiving the resonance frequency corresponding to a specific mRNA sequence, the ribosome is stimulated into initiating the translation process. It starts synthesizing the protein encoded by the mRNA sequence that the resonance frequencies represent.
This process bypasses the traditional need for the physical presence of the mRNA molecule within the cell and instead uses the resonant frequencies as a form of “instruction set” for the ribosome.

### 5. Protein Synthesis:

The ribosome continues to interpret the sequential resonance frequencies as though they were codons on an mRNA molecule. It then synthesizes the corresponding protein until the sequence is complete or until a frequency corresponding to a stop codon is detected.

### 6. Termination and Assessment:

Upon completion, the synthesized protein can be extracted and analyzed to determine if the Mikaelian method effectively guided the ribosome to produce the desired protein.
   This innovative approach not only paves the way for novel therapeutic strategies but also offers a more profound understanding of molecular interactions. By mimicking the natural frequencies of these nucleotides, we can influence cellular processes in a controlled manner, opening new vistas in precision medicine and targeted therapeutics.

By applying these frequencies in a sequence that corresponds to the mRNA's composition, we can essentially “speak” the language of the cell, akin to how ribosomes interpret the mRNA code during protein synthesis. This innovative approach holds immense promise, potentially allowing us to influence cellular activities with unprecedented precision. Through this method, we aim to harness cellular machinery in a manner that aligns with specific therapeutic objectives, heralding a new era in molecular medicine and targeted intervention.

For every nucleotide, namely Adenine (A), Uracil (U) in RNA, Thymine (T), Cytosine (C), and Guanine (G), the Mikaelian Resonance Generator can produce a specific resonance frequency. These frequencies, derived from the Mikaelian Method, emulate the vibrational signatures intrinsic to each nucleotide.

Upon successfully separating the mRNA from the cellular components, our subsequent step utilizes the capabilities of the Mikaelian Resonance Generator. This advanced instrument has been calibrated to simulate the unique resonant frequencies of individual nucleotides present in mRNA sequences.

### Programmed Ribosomes: A New Frontier in Therapeutic and Regenerative Medicine

Programmed ribosomes are bioengineered ribosomes with modified RNA sequences, enabling them to synthesize specific proteins based on the desired outcome.

### Applications

Therapeutic Protein Synthesis: Targeting specific diseases such as cancer, autoimmune disorders, and viral infections (like HIV, hepatitis B, and C).

Halting the synthesis of detrimental proteins in diseases or promoting synthesis of beneficial ones.

Anti-Aging and Cellular Longevity: Synthesizing proteins that can increase the length of telomeres, thereby potentially delaying the aging process at the cellular level.

Tissue Rejuvenation and Regeneration: Producing proteins that promote cellular proliferation in degenerated tissues. Targeting specific tissues that show signs of aging or degeneration, such as skin, muscle, or neural tissues.

Genetic and Cellular Therapies: Working in conjunction with gene-editing techniques to ensure correct protein synthesis post-editing. Clearing or degrading pathological molecules, including amyloid plaques or misfolded proteins.

Drug Delivery and Biotechnology: Being part of therapeutic compositions that can be delivered via various methods, including intravenous injection. Potential bulk production for widespread therapeutic applications.

Research and Database Creation: Generating a database of sequences for programming ribosomes to target various diseases. Developing software algorithms to predict ribosome programming sequences based on desired outcomes.

### Advantages:

Precision and Specificity: Ability to target specific cellular mechanisms or diseases with high specificity.

Flexibility: The reversible programming of ribosomes, allowing them to revert to original functions if needed.

Standardization: Potential for easier regulation due to the direct and controlled production of therapeutic proteins.

Unless otherwise stated, all measurements, values, ratings, positions, magnitudes, sizes, locations, and other specifications that are set forth in this specification, including in the claims that follow, are approximate, not exact. They are intended to have a reasonable range that is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description. These embodiments are capable of modifications in various obvious aspects, all without departing from the spirit and scope of protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing that has been stated or illustrated is intended or should be interpreted to cause a dedication of any component, step, feature, object, benefit, advantage, or equivalent, to the public, regardless of whether it is or is not recited in the claims.

What is claimed is:

1. A method of generating a polypeptide comprising the steps:
providing a sequence of polypeptide;
converting said sequence of polypeptide into a series of a plurality of mRNA codon sequences;
labeling each of said mRNA codon sequences and identifying said mRNA codon sequence based on an order in which said mRNA codon sequences are structured, such that each of said mRNA codon sequences are identified as an nth mRNA codon sequence, wherein n refers to the order in which said mRNA codon sequences are situated pursuant to said series;
identifying a frequency corresponding to each of said mRNA codon sequences;
emitting a series of electromagnetic signals based on said frequency, such that a stimulated ribosome synthesizes the polypeptide as if a physical mRNA having said series of said plurality of mRNA codon sequences; and
generating said polypeptide by said stimulated ribosome.

2. The method of generating a polypeptide of claim 1, wherein said polypeptide is a therapeutic protein.

3. The method of generating a polypeptide: of claim 1 wherein said sequence is designed by humans.

4. The method of generating a polypeptide of claim 1, further comprising the step of tissue regeneration by said polypeptide.

5. The method of generating a polypeptide of claim 1, further comprising the step of increasing lifespan of certain cells by said polypeptide.

6. The method of generating a polypeptide of claim 1, further comprising the step of lengthening telomeres by said polypeptide.

7. A device for stimulating ribosomes, comprising:
an electromagnetic signal transmitter;
wherein said electromagnetic signal transmitter is configured to emit an electromagnetic signal to stimulate a ribosome into synthesizing a polypeptide;
wherein said polypeptide is configured to be translated by said ribosome from a series of codons;
wherein attributes of said electromagnetic signal are calculated based on an analysis of said series of codons; and
wherein said analysis is conducted by converting the molecular properties of individual nucleotides into a frequency, wherein said attributes comprise said frequency.

8. A method of programming ribosomes to synthesize specific proteins by altering their RNA sequence.

9. The method of programming ribosomes of claim 8, wherein said programmed ribosomes are configured to inhibit production of undesirous proteins;
wherein said undesirous proteins may promote tumor growth, as an example.
